(19) United States
(12) Patent Application Publication
(10) Pub. No.: US 2026/0114733 A1
(43) Pub. Date: Apr. 30, 2026

(54) ENHANCED IMAGE FOR NON-CONTACT MONITORING

(71) Applicant: Covidien LP, Mansfield, MA (US)

(72) Inventors: Dean MONTGOMERY, Edinburgh (GB); Paul S. ADDISON, Edinburgh (GB); Dominique JACQUEL, Edinburgh (GB)

(73) Assignee: Covidien LP, Mansfield, MA (US)

(21) Appl. No.: 19/383,619

(22) Filed: Nov. 8, 2025

Related U.S. Application Data
(62) Division of application No. 17/814,946, filed on Jul. 26, 2022, now Pat. No. 12,490,904.
(60) Provisional application No. 63/257,251, filed on Oct. 19, 2021.
(60) Provisional application No. 63/253,953, filed on Oct. 8, 2021.

(51) Int. Cl. A61B 5/00; G06T 7/00; G06T 7/50

(52) U.S. Cl. CPC A61B 5/0077; G06T 7/0012; G06T 7/50; G06T 2207/10028; G06T 2207/30201

(57) ABSTRACT

Methods for enhancing the image of a subject, such as a patient, in a video non-contact monitoring system to provide an enhanced image with clear distinction of the subject from the background. The methods include applying a histogram equalization transform, such as a contrast limited adaptive histogram equalization (CLAHE) transform, to the depth data obtained from a camera of the monitoring system. In some embodiments, the enhanced image of the subject is merged with an overlay image of a monitored physiological parameter determined by the non-contact patient monitoring system.

### CROSS-REFERENCE TO RELATED APPLICATIONS

This application is divisional of U.S. Patent Application No. 17/814,946, filed July 26, 2022, which claims benefit of priority to U.S. Provisional Patent Application No. 63/253,953, filed October 8, 2021, and U.S. Provisional Patent Application No. 63/257,251, filed October 19, 2021, the entire disclosures of which are incorporated herein by reference in their entireties.

### BACKGROUND

Video-based monitoring is a field of patient monitoring that uses a remote video camera to detect physical attributes of the patient, such as respiratory parameters including respiration rate, tidal volume, minute volume, oxygen saturation, effort to breathe, etc., and other patient parameters such as motion and activity, temperature, pulse/heart rate, etc. This type of monitoring may also be called “non-contact” monitoring in reference to the remote video sensor, which does not contact the patient. Many of these parameters are detected and monitored by knowing the distance or depth between the patient surface and a depth sensing camera.

One thing many of these systems have in common is that an image of the patient is provided on a video display and a visual representation of the parameter being monitored is also seen on the display, often overlaid onto the patient image. What is desired is a clear patient image with clear distinction of the patient from the background.

### SUMMARY

The present disclosure is directed to methods for enhancing the image of a subject, such as a patient, in a video non-contact monitoring system to provide a clear image with clear distinction of the subject from the background. The methods include applying a histogram equalization transform, such as a contrast limited adaptive histogram equalization (CLAHE) transform, to the depth data obtained from a camera of the monitoring system. In some embodiments, the enhanced image of the subject is merged with an overlay image of a monitored physiological parameter determined by the non-contact patient monitoring system.

One particular embodiment described herein is a method that includes receiving a video signal having depth data from a non-contact patient monitoring system, enhancing a contrast of the depth data, and applying a colormap to the enhanced contrast depth data to obtain an enhanced image.

Another particular embodiment described herein is a method that includes receiving a video signal having depth data from a non-contact patient monitoring system, extracting a perceptual lightness channel from the video signal, enhancing the contrast of the perceptual lightness channel, merging the perceptual lightness channel back with the channels to obtain an enhanced image, determining an overlay of a monitored physiological parameter by the non-contact patient monitoring system, and merging the enhanced image with the overlay.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Other embodiments are also described and recited herein.

### BRIEF DESCRIPTION OF THE DRAWING

FIG. 1 is a schematic diagram of an example non-contact patient monitoring system according to various embodiments described herein.

FIG. 2A and FIG. 2B are schematic diagrams showing two embodiments using the example non-contact patient monitoring system of FIG. 1.

FIG. 3 is a block diagram of a computing device, a server, and an image capture device according to various embodiments described herein.

FIG. 4A is an image of a patient being monitored by a conventional non-contact patient monitoring system; FIG. 4B is an image of the patient being monitored by the non-contact monitoring system including a background enhancement.

FIG. 5A is an image of a background being monitored by a conventional non-contact patient monitoring system; FIG. 5B is an image of the background being monitored by the non-contact monitoring system including a background enhancement.

FIG. 6 is a stepwise method of an example method for enhancing the visual image from a non-contact patient monitoring system according to various embodiments described herein.

FIG. 7 is a stepwise method of another example method for enhancing the visual image from a non-contact patient monitoring system according to various embodiments described herein.

FIG. 8 is a stepwise method of another example method for enhancing the visual image from a non-contact patient monitoring system according to various embodiments described herein.

FIG. 9 is a stepwise method of another example method for enhancing the visual image from a non-contact patient monitoring system according to various embodiments described herein.

FIG. 10A is an image of a patient being monitored by a conventional non-contact patient monitoring system with a respiratory visual overlay; FIG. 10B is an image of the patient being monitored by the non-contact monitoring system with a respiratory visual overlay including a background enhancement.

FIG. 11 is a stepwise method of an example method for enhancing the visual image from a non-contact patient monitoring system according to various embodiments described herein.

FIG. 12 is a stepwise method of another example method for enhancing the visual image from a non-contact patient monitoring system according to various embodiments described herein.

FIG. 13 is a stepwise method of another example method for enhancing the visual image from a non-contact patient monitoring system according to various embodiments described herein.

FIG. 14 is a stepwise method of another example method for enhancing the visual image from a non-contact patient monitoring system according to various embodiments described herein.

### DETAILED DESCRIPTION

The present disclosure is directed to medical monitoring of a patient, and in particular, non-contact, video-based monitoring of a patient. Systems and methods are described for receiving a video signal view of a patient, identifying a physiologically relevant area within the video image (such as a patient’s forehead or chest), extracting a distance or depth signal from the relevant area, and manipulating the depth data to provide a visual image on a display that is more clear than an image prepared from unmanipulated depth data.

The depth data are detected by a camera or camera system that views but does not contact the patient. With appropriate selection of the data from the signals detected by the camera, a visual image of the patient can be presented with a physiologic parameter of the patient shown in the image. With additional appropriate selection and manipulation of the data, according to the methods herein, the visual image is enhanced, providing an enhanced and more clear image.

Non-contact or remote monitoring, such as video-based monitoring, can deliver significant benefits over contact monitoring. Some video-based monitoring can reduce cost and waste by reducing use of disposable contact sensors, replacing them with reusable camera systems. Video monitoring may also reduce the spread of infection, by reducing physical contact between caregivers and patients. Video cameras can improve patient mobility and comfort, by freeing patients from wired tethers or bulky wearable sensors. In some cases, these systems can also save time for caregivers, who no longer need to reposition, clean, inspect, or replace contact sensors.

One challenge with video monitoring is motion or movement of the patient. Movement in non-contact monitoring creates various complications, due to the extent of movement possible between the patient and the camera. Because the camera is remote from the patient, the patient may move toward or away from the camera, creating a moving frame of reference, or may rotate with respect to the camera, effectively morphing the region that is being monitored. Thus, the monitored tissue can change morphology within the image frame over time.

Another challenge with video monitoring is that the depth data signal received from the camera is, at times, insufficient to provide a clear visual image (e.g., on a display) of the region being monitored. For example, it may be difficult for the viewer of the display to differentiate, e.g., the patient’s head from the pillow from the bed. This may be caused by, e.g., motion noise or other data or signal noise.

The present disclosure describes methods for enhancing the visual distinction of the patient and/or the background for non-contact monitoring of a patient to determine physiological parameter(s) such as respiration rate, tidal volume, minute volume, oxygen saturation, temperature, pulse/heart rate, motion and activity, etc. The systems and methods receive a video signal from the patient and from that extract distance or depth data from the relevant area and then manipulate the data to provide an enhanced image.

The depth sensing feature provides a measurement of the distance or depth between the detection system and the patient. One or two video cameras may be used to determine the depth, and change in depth, from the system to the patient. When two cameras, set at a fixed distance apart, are used, they offer stereo vision due to the slightly different perspectives of the scene from which distance information is extracted. When distinct features are present in the scene, the stereo image algorithm can find the locations of the same features in the two image streams. However, if an object is featureless (e.g., a smooth surface with a monochromatic color), then the depth camera system has difficulty resolving the perspective differences. By including an image projector to project features (e.g., in the form of dots, pixels, etc.) onto the scene, this projected feature can be monitored over time to produce an estimate of changing distance or depth.

In the following description, reference is made to the accompanying drawing that forms a part hereof and in which is shown by way of illustration at least one specific embodiment. The following description provides additional specific embodiments. It is to be understood that other embodiments are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples, including the figures, provided below. In some instances, a reference numeral may have an associated sub-label consisting of a lower-case letter to denote one of multiple similar components. When reference is made to a reference numeral without specification of a sub-label, the reference is intended to refer to all such multiple similar components.

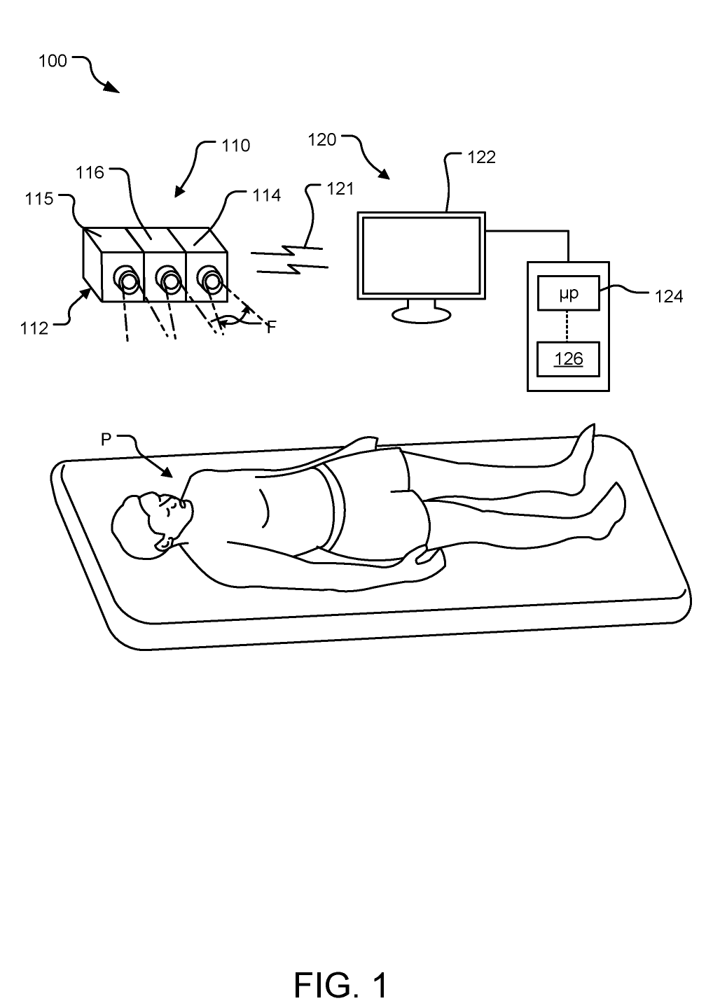

FIG. 1 shows a non-contact patient monitoring system 100 and a patient P. The system 100 includes a non-contact detector system 110 placed remote from the patient P. In this embodiment, the detector system 110 includes a camera system 112, particularly, that includes an infrared (IR) detection feature. The camera system 112 includes a first camera 114 and a second camera 115, at least one of which is a depth sensing camera, such as a Kinect camera from Microsoft Corp. (Redmond, Washington) or a RealSense^{TM} D415, D435 or D455 camera from Intel Corp. (Santa Clara, California).

The cameras 114, 115 are positioned so that their ROI at least intersect, in some embodiments overlap. The detector system 110 also includes an IR projector 116, which projects individual features (e.g., dots, crosses or Xs, lines, or a featureless pattern, or a combination thereof etc.) onto the ROI. The projector 116 can be separate from the camera system 112 or integral with the camera system 112, as shown in FIG. 1. In some embodiments, more than one projector 116 can be used. Both cameras 114, 115 and the projector 116 are aimed to have the features projected by the projector 116 to be in the ROI.

The cameras 114, 115 and projector 116 are remote from the patient P, in that they are spaced apart from and do not contact the patient P. The camera system 112 includes a detector exposed to a field of view F that encompasses at least a portion of the patient P.

The camera system 112 includes at least one depth sensing camera, either or both camera 114 or camera 115, that can detect a distance between the camera system 112 and objects in its field of view F. Such information can be used to determine that a patient is within the field of view of the camera system 112 and determine a region of interest (ROI) to monitor on the patient. Once an ROI is identified, that ROI can be monitored over time, and the change in depth of points within the ROI can represent movements of the patient associated with, e.g., breathing. Accordingly, those movements, or changes of depth points within the ROI, can be used to determine, e.g., respiration rate, tidal volume, minute volume, effort to breathe, etc.

In some embodiments, the field of view F encompasses exposed skin of the patient. In other embodiments, the field of view F encompasses a portion of the patient’s torso, covered by a blanket, sheet, or gown.

The cameras 114, 115 operate at a frame rate, which is the number of image frames taken per second (or other time period). Example frame rates include 15, 20, 30, 40, 50, or 60 frames per second, greater than 60 frames per second, or other values between those. Frame rates of 20-30 frames per second produce useful signals, though frame rates above 100 or 120 frames per second are helpful in avoiding aliasing with light flicker (for artificial lights having frequencies around 50 or 60 Hz).

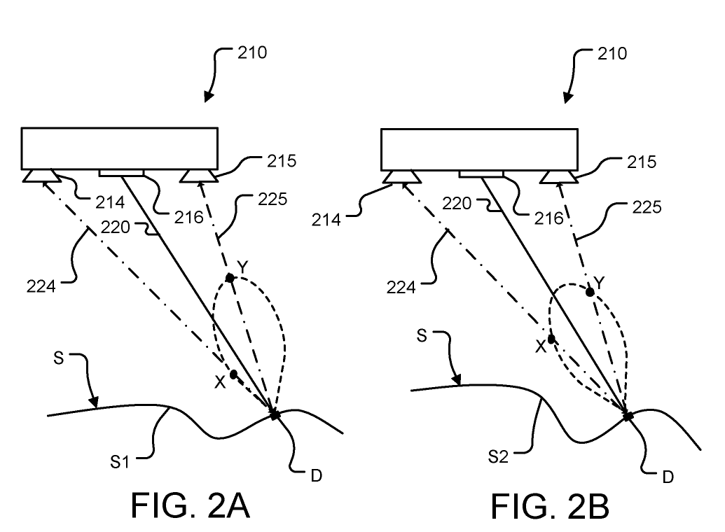

The distance from the ROI on the patient P to the camera system 112 is measured by the system 100. Generally, the camera system 112 detects a distance between the camera system 112 and the projected features on the surface of the patient P within the ROI; the change in depth or distance of the ROI represents movements of the patient P, e.g., associated with breathing. The light from the projector 116 hitting the surface is scattered/diffused in all directions and is monitored by the camera system 112 to determine the distance; the diffusion pattern depends on the reflective and scattering properties of the surface. The camera system 112 also detects the light intensity of the projected individual features in their ROIs. From the distance and the light intensity, at least one physiological parameter of the patient P is monitored. Additional details are provided below in respect to FIG. 2A and FIG. 2B.

Different methods can be used to identify the patient and define an ROI. In some embodiments, the system 100 determines a skeleton outline of the patient P to identify a point or points from which to extrapolate the ROI. For example, a skeleton may be used to find a center point of a chest, shoulder points, waist points, and/or any other points on a body. These points can be used to determine the ROI. For example, the ROI may be defined by filling in the area around a center point of the chest. Certain determined points may define an outer edge of an ROI, such as shoulder points. In other embodiments, instead of using a skeleton, other points are used to establish an ROI. For example, a face may be recognized, and a chest area inferred in proportion and spatial relation to the face. In other embodiments, the system 100 may establish the ROI around a point based on which parts are within a certain depth range of the point. In other words, once a point is determined that an ROI should be developed from, the system can utilize the depth information from the depth sensing camera system 112 to fill out the ROI as disclosed herein. For example, if a point on the chest is selected, depth information is utilized to determine the ROI area around the determined point that is a similar distance from the depth sensing camera 114 as the determined point. This area is likely to be a chest.

The ROI size may differ according to the distance of the patient from the camera system. The ROI dimensions may vary linearly with the distance of the patient from the camera system. This ensures that the ROI scales according with the patient and covers the same part of the patient regardless of the patient’s distance from the camera. This is accomplished by applying a scaling factor that is dependent on the distance of the patient (and the ROI) from the camera. In order to properly measure the depth changes, the actual size (area) of the ROI is determined and movements of that ROI are measured. The measured movements of the ROI and the actual size of the ROI are then used to calculate a parameter, e.g., a tidal volume. Because a patient’s distance from a camera can change, e.g., due to the patient’s rolling or position readjustment, the ROI associated with that patient can appear to change in size in an image from a camera. However, using the depth sensing information captured by a depth sensing camera or other type of depth sensor, the system can determine how far away from the camera the patient (and their ROI) actually is. With this information, the actual size of the ROI can be determined, allowing for accurate measurements of depth change regardless of the distance of the camera to the patient.

In some embodiments, the system 100 may receive a user input to identify a starting point for defining an ROI. For example, an image may be reproduced on an interface, allowing a user of the interface to select a patient for monitoring (which may be helpful where multiple humans are in view of a camera) and/or allowing the user to select a point on the patient from which the ROI can be determined (such as a point on the chest). Other methods for identifying a patient, points on the patient, and defining an ROI may also be used.

To determine the distance from the camera system 112 and the projected image on the patient P, the detected images and diffusion measurements (detected by the camera system 112) are sent to a computing device 120 through a wired or wireless connection 121. The computing device 120 includes a display 122, a processor 124, and hardware memory 126 for storing software and computer instructions. Sequential image frames of the patient P are recorded by the video camera system 112 and sent to the processor 124 for analysis. The display 122 may be remote from the camera system 112, such as a video screen positioned separately from the processor and memory. Other embodiments of the computing device 120 may have different, fewer, or additional components than shown in FIG. 1. In some embodiments, the computing device may be a server. In other embodiments, the computing device of FIG. 1 may be additionally connected to a server. The captured images (e.g., still images, or video) can be processed or analyzed at the computing device and/or at the server to determine the parameters of the patient P as disclosed herein.

FIG. 2A and FIG. 2B both show a non-contact detector 210 having a first camera 214, a second camera 215, and an IR projector 216. A dot D is projected by the projector 216 onto a surface S, e.g., of a patient, via a beam 220. Light from the dot D is reflected by the surface S and is detected by the camera 214 as beam 224 and by the camera 215 as beam 225.

In a particular implementation, the light intensity returned to and observed by the cameras 214, 215 depends on the diffusion pattern caused by the surface S (e.g., the surface of a patient), the distance between the cameras 214, 215 and surface S, the surface gradient, and the orientation of the cameras 214, 215 relative to the surface S. In FIG. 2A, the surface S has a first profile S1 and in FIG. 2B, the surface S has a second profile S2 different than S1; as an example, the first profile S1 is during an exhale breath of a patient and the second profile S2 is during an inhale breath of the patient. Because the surface profiles S1 and S2 differ, the deflection pattern from the dot D on each of the surfaces differs for the two figures, and hence the distance from the cameras 214, 215 to the surface differs for the two figures.

During movement such as breathing, the light intensity reflection off the dot D observed by the cameras 214, 215 changes because the surface profile S1 and S2 (specifically, the gradient) changes as well as the distance between the surface S and the cameras 214, 215. FIG. 2A shows the surface S having the surface profile S1 at time instant t=tₙ and FIG. 2B shows the surface S having the surface profile S2 at a later time, specifically t=tₙ₊₁,with S2 being slightly changed due to motion caused by respiration. Consequently, the intensity of the projected dot D observed by the cameras 214, 215 will changed due to the changes of the surface S. In FIG. 2A, a significantly greater intensity is measured by the camera 215 than the camera 214, seen by the x and y on the beams 224, 225, respectively. In FIG. 2B, y is less than y in FIG. 2A, whereas x in FIG. 2B is greater than x in FIG. 2A. The manner in how these intensities change depends on the diffusion pattern and its change over time, which are related to movement of the surface S. As seen in FIGS. 2A and 2B, the light intensities as measured by the cameras 214, 215 have changed between FIGS. 2A and 2B, and hence, the surface S has moved. Each camera will generate a signal because of the change of the intensity of dot D when the surface profile changes from time instant t=tₙ to t=tₙ₊₁ due to movement.

In some other embodiments, a single camera and light projector can be used. For example, the camera 215 may be not present or is ignored. It is clear that the camera 214 will still produce a change in light intensity from time instant t=tₙ to t=tₙ₊₁ due to movement. This embodiment will therefore produce only a single signal as opposed to the two signals generated by the embodiment discussed in the previous paragraph.

Alternatively, other depth camera detectors may be used for the monitoring system. For example, the depth camera detector and/or the depth camera(s) may be based on, for example, stereoscopic, structured light, or time-of-flight principles.

Stereoscopic depth cameras resolve depth by using two slightly different perspective views of the same scene, similar to the detector 210 of FIGS. 2A and 2B; this is similar to the manner in which frontal vision animals perceive depth. Algorithmically, the depth data is constructed from the two views by calculating the disparities between features or key points in the scene.

Structured light and related coded light-based cameras project a pattern (e.g., an IR pattern) onto a scene; the pattern, which may be a series of stripes or dots, for example, has a known visual shape. Depth data is obtained by analyzing the deformation of the shape perceived by the camera, the deformation due to the movement of the scene. This detected movement is correlated to the distance from the cameras to the deformed pattern on the scene.

Depth cameras operate on the time-of-flight principle and measure distance (depth) to points in the scene by measuring the time it takes for a signal emitted from the camera to return due to reflection from a surface. The scene is actively illuminated by the camera’s emitter (e.g., a radiation emitter, such as an IR laser) and the camera recovers the distance information either through a direct (i.e., half the return time) or indirect (i.e., phase recovery of a modulated emitted signal) method.

In addition to the methods and cameras/detectors described above, any suitable method for determining depth data from a scene can be used in the methods described herein.

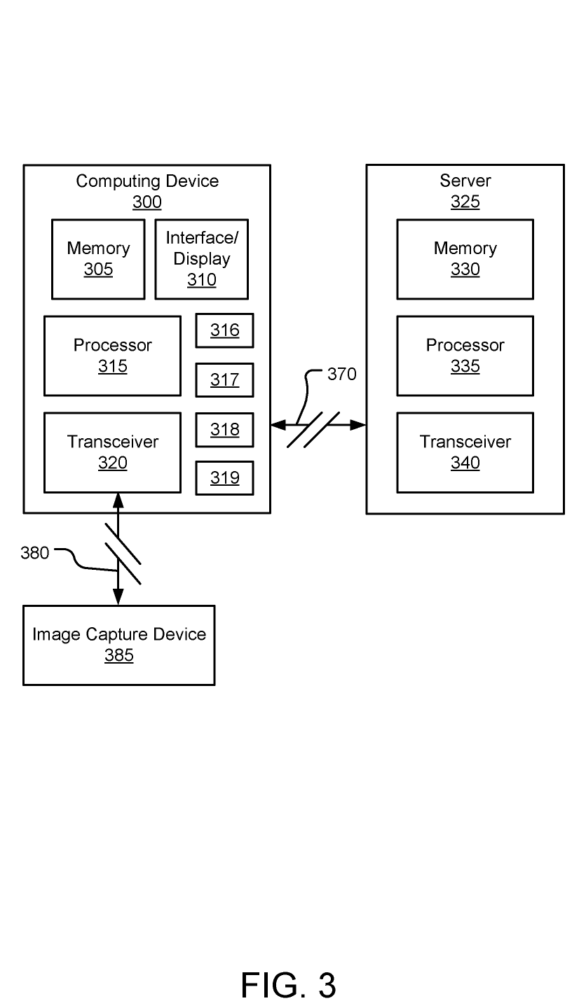

FIG. 3 is a block diagram illustrating a system including a computing device 300, a server 325, and an image capture device 385 (e.g., a camera, e.g., the camera system 112 or cameras 114, 115). In various embodiments, fewer, additional and/or different components may be used in the system.

The computing device 300 includes a processor 315 that is coupled to a memory 305 to store and recall data and applications in the memory 305, including applications that process information and send commands/signals according to any of the methods disclosed herein. The computing device 300 includes, in this example, modules 316, 317, 318 and 319, each configured to execute one or more of the analytical methods for manipulating the depth data described below to determine the enhanced image.

The processor 315 may also display objects, applications, data, etc. on an interface/display 310. The processor 315 may also or alternately receive inputs through the interface/display 310. The processor 315 is also coupled to a transceiver 320. With this configuration, the processor 315, and subsequently the computing device 300, can communicate with other devices, such as the server 325 through a connection 370 and the image capture device 385 through a connection 380. For example, the computing device 300 may send to the server 325 information determined about a patient from images captured by the image capture device 385, such as depth information of a patient in an image.

The server 325 also includes a processor 335 that is coupled to a memory 330 and to a transceiver 340. The processor 335 can store and recall data and applications in the memory 330. In some implementations, the server 325 may include the modules for manipulating the depth data, rather than the computing device 300. With this configuration, the processor 335, and subsequently the server 325, can communicate with other devices, such as the computing device 300 through the connection 370.

The computing device 300 may be, e.g., the computing device 120 of FIG. 1. Accordingly, the computing device 300 may be located remotely from the image capture device 385, or it may be local and close to the image capture device 385 (e.g., in the same room). The processor 315 of the computing device 300 may perform any or all of the various steps disclosed herein. In other embodiments, the steps may be performed on a processor 335 of the server 325. In some embodiments, the various steps and methods disclosed herein may be performed by both of the processors 315 and 335. In some other embodiments, certain steps may be performed by the processor 315 while others are performed by the processor 335. Information determined by the processor 315 may be sent to the server 325 for storage and/or further processing.

The devices may be utilized in various ways. For example, either or both of the connections 370, 380 may be varied. For example, either or both the connections 370, 380 may be a hard-wired connection. A hard-wired connection may involve connecting the devices through a USB (universal serial bus) port, serial port, parallel port, or other type of wired connection to facilitate the transfer of data and information between a processor of a device and a second processor of a second device. In another example, one or both of the connections 370, 380 may be a dock where one device may plug into another device. As another example, one or both of the connections 370, 380 may be a wireless connection. These connections may be any sort of wireless connection, including, but not limited to, Bluetooth connectivity, Wi-Fi connectivity, infrared, visible light, radio frequency (RF) signals, or other wireless protocols/methods. For example, other possible modes of wireless communication may include near-field communications, such as passive radio-frequency identification (RFID) and active RFID technologies. RFID and similar near-field communications may allow the various devices to communicate in short range when they are placed proximate to one another. In yet another example, the various devices may connect through an internet (or other network) connection. That is, one or both of the connections 370, 380 may represent several different computing devices and network components that allow the various devices to communicate through the internet, either through a hard-wired or wireless connection. One or both of the connections 370, 380 may also be a combination of several modes of connection.

The configuration of the devices in FIG. 3 is merely one physical system on which the disclosed embodiments may be executed. Other configurations of the devices shown may exist to practice the disclosed embodiments as well as configurations of additional or fewer devices than the ones shown in FIG. 3. Additionally, any of the devices shown in FIG. 3 may be combined to allow for fewer devices than shown or separated such that more than the three devices exist in a system. It will be appreciated that many various combinations of computing devices may execute the methods and systems disclosed herein. Examples of such computing devices may include other types of medical devices and sensors, infrared cameras/detectors, night vision cameras/detectors, other types of cameras, radio frequency transmitters/receivers, smart phones, personal computers, servers, laptop computers, tablets, RFID enabled devices, or any combinations of such devices.

The methods of this disclosure utilizes depth (distance) data between the camera(s) and the patient to produce a visual image of the patient and the background, the image being produced by applying a histogram equalization transform to the depth data.

A depth image or depth map, which includes information about the distance from the camera(s) to each point in the image, can be measured or otherwise captured by a depth sensing camera, such as a Kinect camera from Microsoft Corp. (Redmond, Washington) or a RealSense^{TM} D415, D435 or D455 camera from Intel Corp. (Santa Clara, California) or other sensor devices based upon, for example, millimeter wave and acoustic principles to measure distance. The depth image or map can be obtained by a stereo camera, a camera cluster, camera array, or a motion sensor focused on a ROI, such as a patient’s chest. In some embodiments, the camera(s) are focused on visible or IR features in the ROI. Each projected feature may be monitored, less than all the features in the ROI may be monitored or all the pixels in the ROI can be monitored.

Because the image includes depth data or a depth map from the depth sensing camera(s), information on the spatial location of the patient (e.g., the patient’s chest) in the ROI can be determined. For example, as the patient breathes, the patient’s chest moves toward and away from the camera, changing the depth information associated with the images over time. As a result, the location information associated with the ROI changes over time. For example, movement of a patient’s chest toward the camera as the patient’s chest expands forward represents inhalation. Similarly, movement backward, away from the camera, occurs when the patient’s chest contracts with exhalation. This movement forward and backward can be tracked to determine a respiration rate.

The non-contact monitoring system (e.g., system 100 of FIG. 1) utilizes the display (e.g., display 122 of FIG. 1) to provide an image of the patient to the viewer overlayed with the monitored physiological parameter, e.g., respiration rate. If the depth data or depth map is displayed in a generally-unaltered format, the image can be fairly grainy and nondescript, with the patient often not distinguishable from the background. By applying a histogram equalization (HE) transform, such as a contrast limited adaptive histogram equalization (CLAHE) transform, to the depth data, the resulting image has better contrast so that the patient and details can be better viewed, and the resulting image may have smoother transitions.

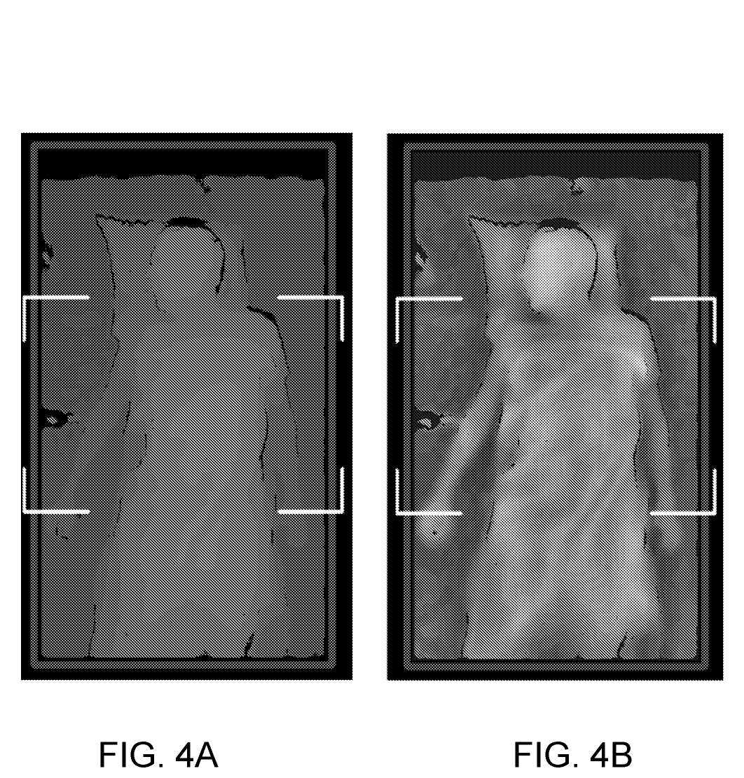

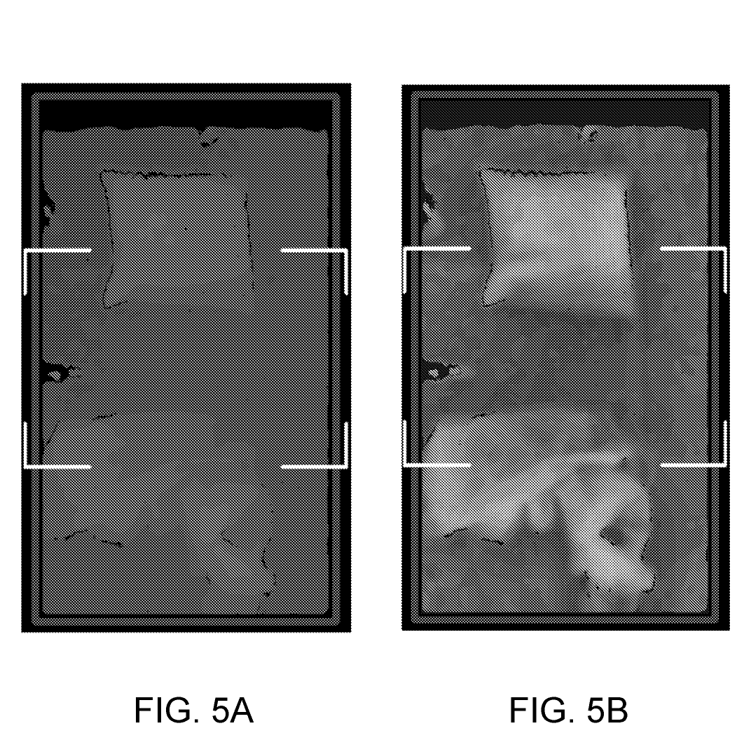

FIGS. 4A and 4B and FIGS. 5A and 5B show the visual benefit obtained by applying the histogram equalization transform to the depth data. FIGS. 4A and 4B show a patient on a bed with a pillow, whereas FIGS. 5A and 5B show the bed with the pillow but with the patient no longer in the scene. In both FIGS. 4B and 5B, the enhanced images, as an example, the folds and wrinkles of the blanket can be readily identified.

In FIG. 4A and in FIG. 5A, the images are standard images obtained from the raw depth data obtained from a non-contact monitoring system using a RealSense D415 camera. FIG. 4B and FIG. 5B show images obtained from the same depth data with a CLAHE transform applied to the raw depth data. It is noted that the enhancement to the image is across the entire image, not just in the region of interest (ROI), shown in the brackets, which is monitored for the physiological parameter.

It is also noted that although the images provided herein show a bed (e.g., a hospital bed) either empty or with a lying patient thereon, the non-contact monitoring may be focused on a seated patient (e.g., seated in a bed or on a chair), on a standing patient, or a patient in any other position and/or location.

The data manipulation, overall, includes applying a histogram equalization transform to the depth data to flatten, smooth, and/or filter the histogram of the data. A colormap can be applied to provide color contrast. By utilizing the adaptive equalization, such as of CLAHE transform, in the manner as describe herein, the color scale of the raw data image is maintained while providing a high contrast in areas where there is a large change in the depth data.

In some methods, the data manipulation includes decomposing the raw data image into a different colorspace (the colorspace having three channels), applying the histogram equalization transform to flatten, smooth and/or filter the histogram, and then merging the three channels back together to the original colorspace. The histogram equalization transform is applied to the perceptual lightness (e.g., luminosity or other light or brightness aspect) of the colorspace. To enhance the image from the depth data, it is the perceptual lightness aspect that is optimized. By utilizing the adaptive equalization of the transform in the manner as described herein, the grey scale of the raw data image is maintained while providing a high contrast in areas where there is a large change in the depth data.

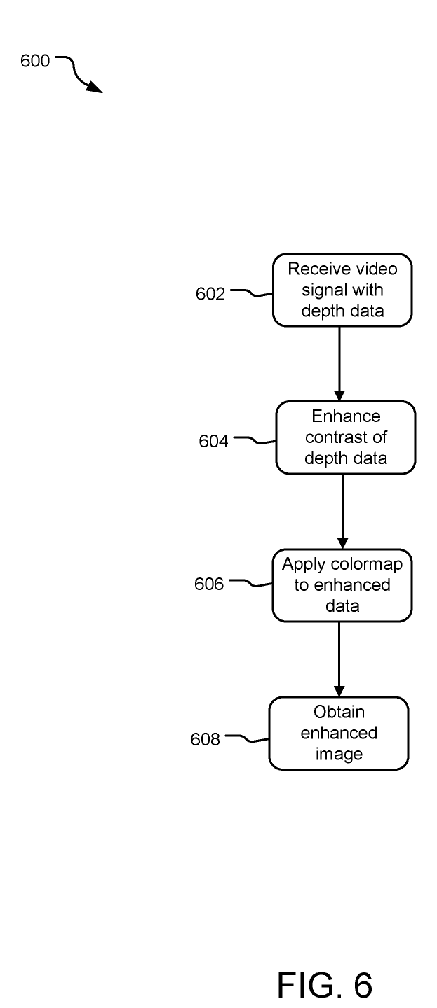

FIG. 6 shows, stepwise, an overall method 600 for manipulating depth data to enhance the resultant visual image, whether the image is the patient and background (as in FIG. 4B) or only background (as in FIG. 5B).

In step 602, a video signal that includes depth data is received from camera(s) of a non-contact monitoring system for a region of interest, which typically includes a patient. The monitoring is done via depth data obtained from depth camera(s) based on the distance of the patient or other surface in relation to the depth camera(s). In step 604, the contrast of the depth data is enhanced, e.g., transformed, to apply a visual depth indication. In step 606, a colormap is applied to the enhanced data. In step 608, an enhanced image is obtained, enhanced in details in relation to an image from the original, untransformed, depth data.

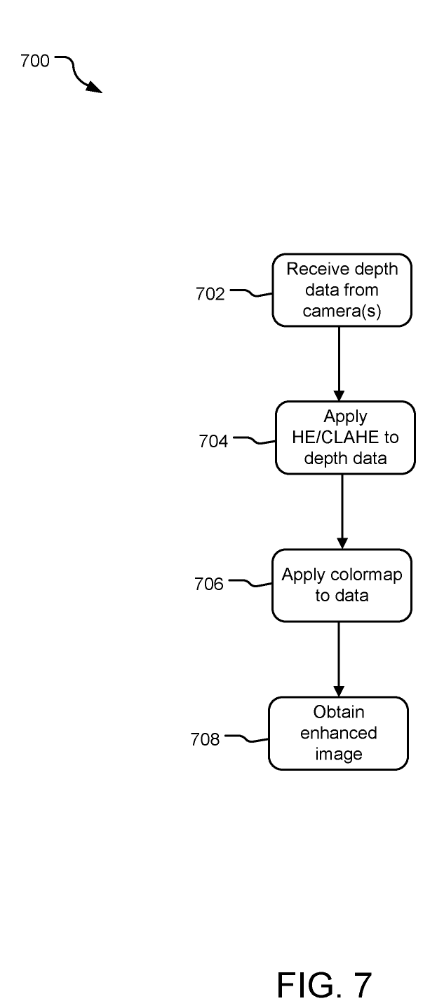

Another overall method 700, for manipulating depth data to enhance the resultant visual image, whether the image is the patient and background (as in FIG. 4B) or only background (as in FIG. 5B), is shown in FIG. 7.

In step 702, a video signal that includes depth data is received from camera(s) of a non-contact monitoring system for a region of interest, which typically includes a patient. The monitoring is done via depth data obtained from depth camera(s) based on the distance of the patient or other surface in relation to the depth camera(s). In step 704, a histogram equalization (HE) transform, such as a CLAHE transform, is applied to the depth data to enhance the depth data. In step 706, a colormap is applied to the transformed (e.g., flattened, smoothed, filtered) data, to apply a visual depth indication. In step 708, an enhanced image is obtained, enhanced in details in relation to an image from the original, untransformed, depth data from the video signal.

In one example method, the transformed depth data is converted to a color image by way of applying a colormap to provide a visual distinction of the depth, due to changes in color proportional to the transformed depth data. Any colormap suitable for the data is acceptable. Examples of suitable colormaps include “bone,” “viridis,” “parula,” “jet,” etc.

In some embodiments, two different colors or colormaps may be used for the enhanced image. For example, a bed may be shown in a different color or colormap than a patient; this may be accomplished by applying one color to objects that are closer to the camera(s) (e.g., a patient) and another color to objects that are farther away (e.g., the bed on which the patient is laying). In some embodiments, a different color or colormap can be limited to a distinct area, e.g., outlined area, such as a patient’s chest or face. Medical devices, e.g., tubing, leads, sensors, etc., may be shown in a different color or colormap.

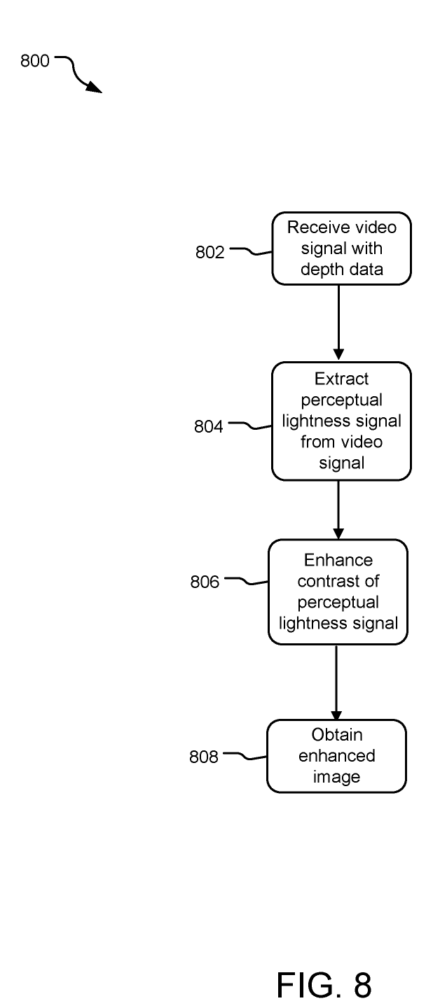

FIG. 8 shows, stepwise, another overall method 800 for manipulating depth data to enhance the resultant visual image, whether the image being the patient and background (as in FIG. 4B) or only background (as in FIG. 5B).

In step 802, a video signal that includes depth data is received from camera(s) of a non-contact monitoring system for a region of interest, which typically includes a patient. The monitoring is done via depth data obtained from depth camera(s) based on the distance of the patient or other surface in relation to the depth camera(s). In step 804, the perceptual lightness (e.g., luminosity) signal is extracted from the video signal, and in step 806 the contrast of the signal is enhanced. In step 808, an enhanced image is obtained, enhanced in details in relation to an image from the original, untransformed, depth data from the video signal.

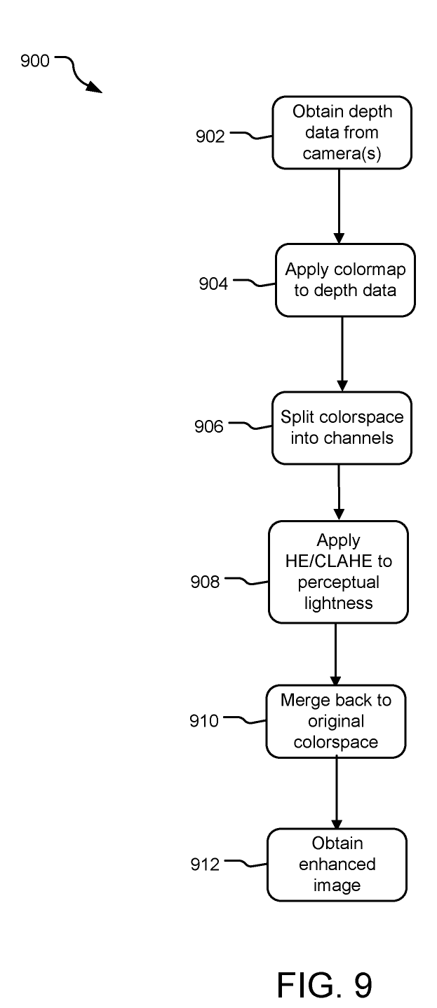

FIG. 9 shows, stepwise, another overall method 800 for manipulating depth data to enhance the resultant visual image, whether the image being the patient and background (as in FIG. 4B) or only background (as in FIG. 5B).

In step 902, a video signal that includes depth data is received from camera(s) of a non-contact monitoring system for a region of interest, which typically includes a patient. The monitoring is done via depth data obtained from depth camera(s) based on the distance of the patient or other surface in relation to the depth camera(s). In step 904, a colormap is applied to the depth data. In step 906, the three channels of the colormapped-depth are split, with one of the channels being perceptual lightness (e.g., luminosity, lightness, brightness, or similar). In step 908, a CLAHE transform is applied to the perceptual lightness channel of the colorspace. In step 910, the channels are merged back to the original colorspace. In step 912, an enhanced image is obtained, enhanced in details in relation to an image from the original, untransformed, depth data.

In one example method, the depth data is obtained in RGB colorspace (having channels: red and green and blue). The image is decomposed into LAB colorspace (having channels: luminosity and green-red and blue-yellow). A CLAHE transform is applied to the luminosity aspect, to flatten, smooth and/or filter the luminosity histogram, thus adding contrast to the channel. The LAB channels are converted back to RGB, so that the final image is back to RGB colorspace.

Other examples of suitable colorspaces include HSL (having channels: hue and saturation and lightness or luminance), HSV (having channels: hue and saturation and value), HSB (having channels: hue and saturation and brightness), and others such as cylindrical transformation, YCbCr (channels: luma and blue-difference and red-difference), YUV, and subtractive CMYK (cyan, magenta, yellow, black) and CMY (cyan, magenta, yellow).

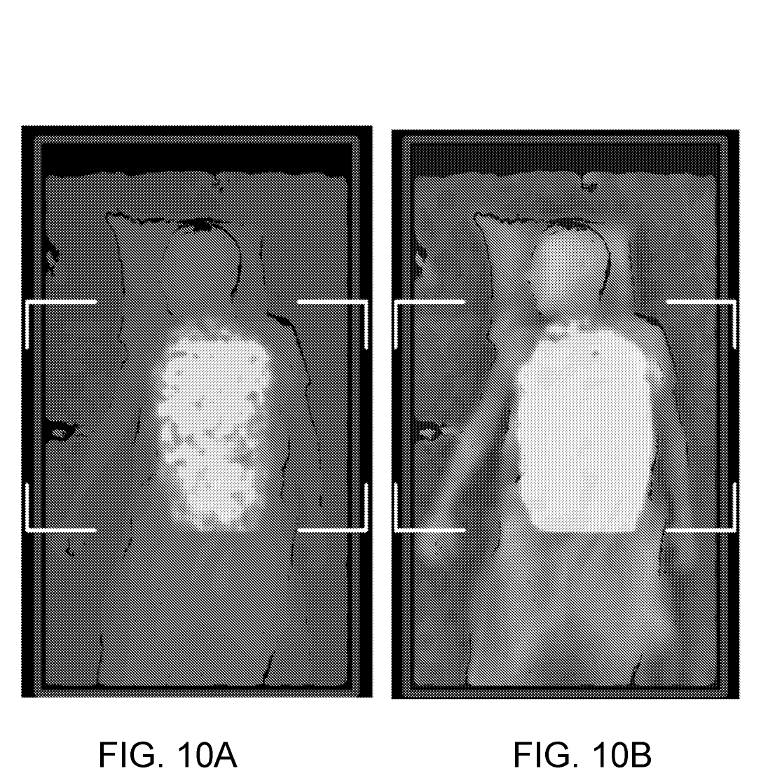

FIGS. 10A and 10B also show the visual benefit obtained by applying the CLAHE transform to the depth data. In these figures, the background image, which includes the patient, has been overlaid with a visual representation of the parameter being monitoring, which in this example, is respiration, particularly, a visual representation of inhalation. The visual representation of inhalation is obtained, as described above, by the change in distance of the patient’s chest to the camera(s).

In FIG. 10A, the image is a standard image obtained from the raw depth data obtained from a non-contact monitoring system and overlayed with the respiration representation also obtained from the raw depth data. FIG. 10B shows the image obtained from the same depth data with the CLAHE transform applied to the raw depth data for the background and the patient overlayed with the respiration representation obtained from the raw depth data. It is noted that the enhancement to the image is across the entire image, not just in the region of interest (ROI), shown in the brackets, which is monitored for the respiration parameter.

Depending on the system parameters of the non-contact monitoring system being used for the monitoring of the physiological parameter (respiration, in this example), the forward and backward movement of the patient’s chest is evidenced by a color change applied by the monitoring system. For example, when the ROI region is moving towards the camera (e.g., on an inhale), a green overlay can be shown, whereas when the ROI region is moving away from the camera (e.g., on an exhale), no color overlay is shown. In other implementations, the user or viewer of the monitoring system can select the settings of the visual output. For example, the user may desire a green overlay for an inhale and a red overlay for an exhale, or, a white overlay for an inhale and no color overlay for an exhale, e.g., for user that are red/green colorblind. In some arrangements, the strength, tone, or brightness of the selected color may change as the movement (e.g., distance) changes.

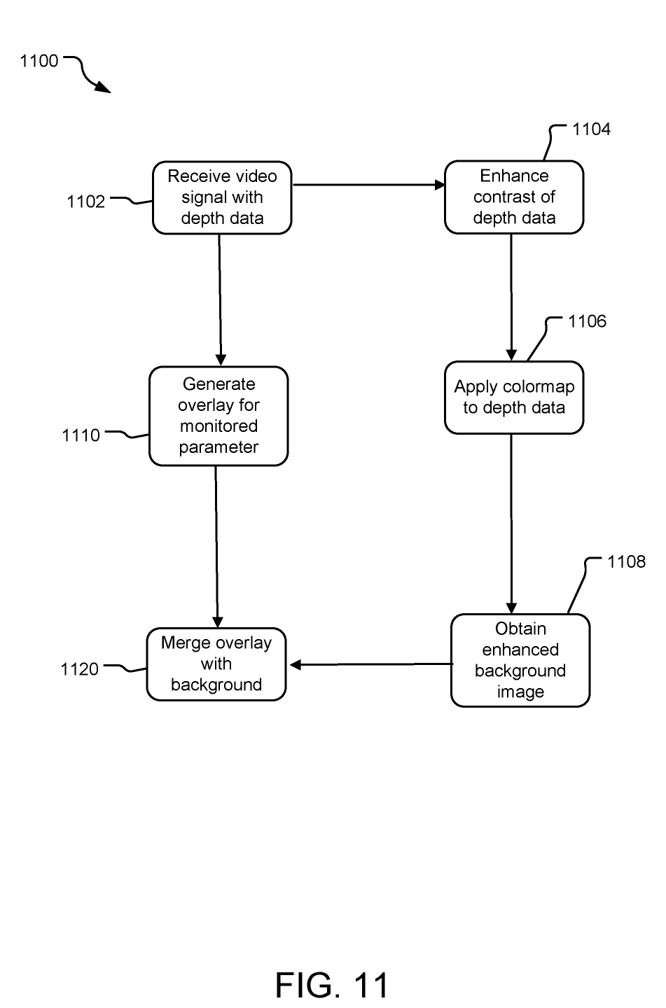

FIG. 11 shows, stepwise, another overall method 1100 for manipulating depth data to enhance the resultant visual image. This method 1100 enhances the background and/or the patient and combines the enhanced image with a monitored physiological parameter (as in FIG. 10B).

In step 1102, a video signal that includes depth data is received from camera(s) of a non-contact monitoring system for a region of interest, which typically includes a patient. The monitoring is done via depth data obtained from depth camera(s) based on the distance of the patient or other surface in relation to the depth camera(s). In step 1104, the contrast of the depth data from step 1102 is enhanced; this enhanced data thus has a colormap applied thereto in step 1106. With the colormap applied, an enhanced background image is obtained in step 1108.

Prior to, subsequent to, or simultaneous to applying steps 1104 through 1108, the depth data from step 1102 is used to generate a visual overlay for the monitored physiological parameter, such as respiration, in step 1110.

In step 1120, the visual image of the parameter from step 1110 is merged with the enhanced background image from step 1108.

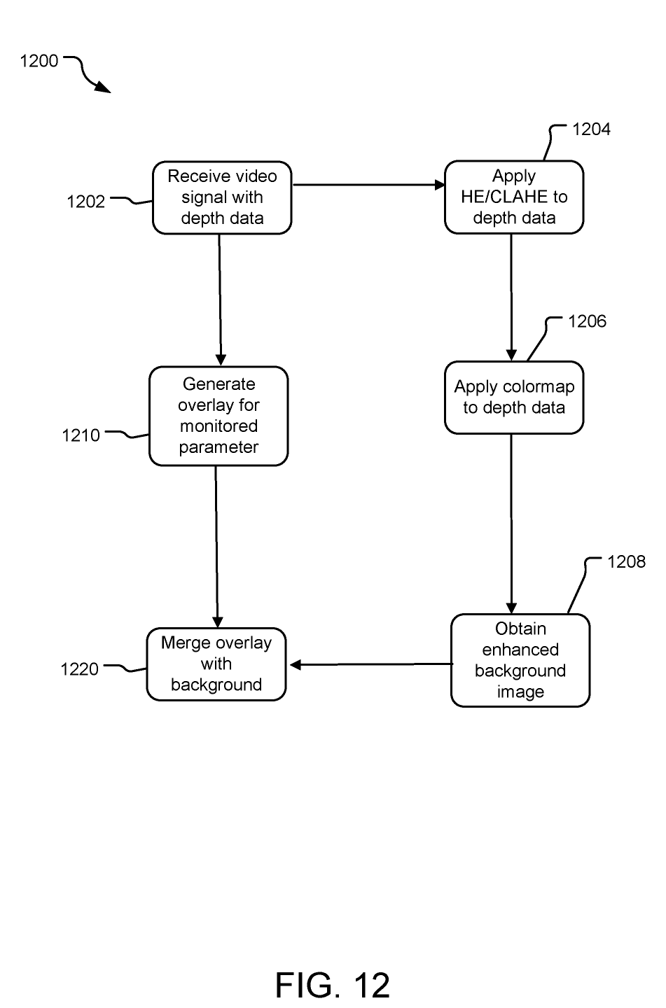

FIG. 12 shows, stepwise, another overall method 1200 for manipulating depth data to enhance the resultant visual image. This method 1200 enhances the background and/or the patient and combines the enhanced image with a monitored physiological parameter (as in FIG. 10B).

In step 1202, a video signal that includes depth data is received from camera(s) of a non-contact monitoring system for a region of interest, which typically includes a patient. The monitoring is done via depth data obtained from depth camera(s) based on the distance of the patient or other surface in relation to the depth camera(s). In step 1204, a histogram equalization (HE) (e.g., CLAHE) transform is applied to the depth data from step 1202; this flattened data thus has a colormap applied thereto in step 1206. With the colormap applied, an enhanced background image is obtained in step 1208.

Prior to, subsequent to, or simultaneous to applying steps 1204 through 1208, the depth data from step 1202 is used to determine the monitored physiological parameter, such as respiration, in step 1210.

In step 1220, the visual image of the parameter from step 1210 is merged with the enhanced background image from step 1208.

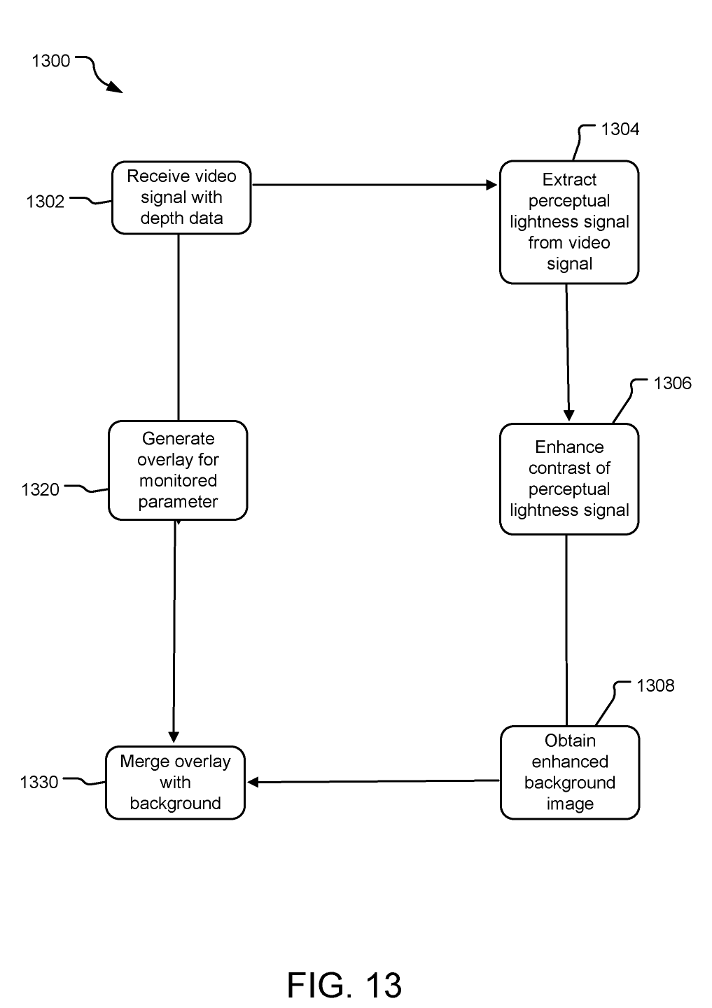

FIG. 13 shows, stepwise, another overall method 1300 for manipulating depth data to enhance the resultant visual image. This method 1300 enhances the background and/or the patient and combines the enhanced image with a monitored physiological parameter (as in FIG. 10B).

In step 1302, a video signal that includes depth data is received from camera(s) of a non-contact monitoring system for a region of interest, which typically includes a patient. The monitoring is done via depth data obtained from depth camera(s) based on the distance of the patient or other surface in relation to the depth camera(s).

In step 1304, the perceptual lightness signal is extracted from the video signal and in step 1306, the contrast of the perceptual lightness signal is enhanced. From the enhanced perceptual lightness signal, an enhanced background image is obtained in step 1308.

Prior to, subsequent to, or simultaneous to applying steps 1304 through 1308, the depth data from step 1302 is used to generate a visual overlay representative of a monitored physiological parameter, such as respiration, in step 1320.

In step 1330, the visual overlay of the parameter from step 1320 is merged with the enhanced background image from step 1308.

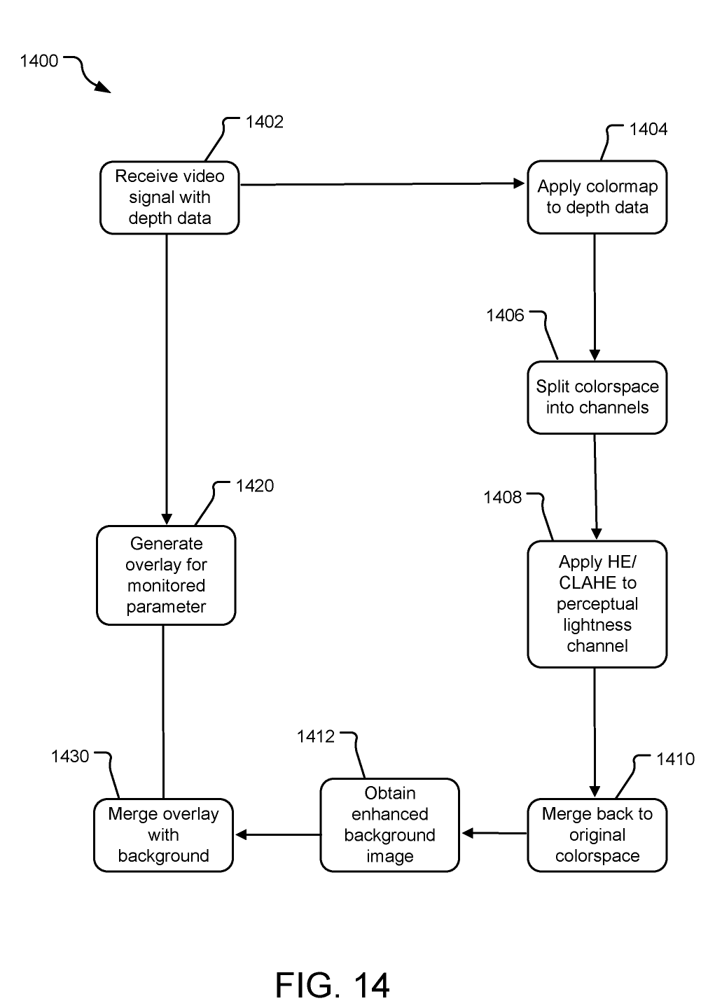

FIG. 14 shows, stepwise, yet another overall method 1400 for manipulating depth data to enhance the resultant visual image.

In step 1402, a video signal that includes depth data is received from camera(s) of a non-contact monitoring system for a region of interest, which typically includes a patient.

In step 1404, a colormap is applied to the depth data. In step 1406, the three channels of the colormapped-depth are split, with one of the channels being perceptual lightness (e.g., luminosity, lightness, brightness, or similar). In step 1408, an HE (e.g., CLAHE) transform is applied to the perceptual lightness channel. In step 1410, the channels are merged back to the original colorspace, resulting in the enhanced background image in step 1412.

Prior to, subsequent to, or simultaneous to applying steps 1404 through 1412, the depth data from step 1402 is used to generate a visual overlay of the monitored physiological parameter, such as respiration, in step 1420.

In step 1430, the visual overlay image of the parameter from step 1420 is merged with the enhanced background image from step 1412.

In some embodiments, two different colors or colormaps may be used for the enhanced image. For example, a bed may be shown in a different color or colormap than a patient; this may be accomplished by applying one color to objects that are closer to the camera(s) (e.g., a patient) and another color to objects that are farther away (e.g., the bed on which the patient is laying). In some embodiments, a different color or colormap can be limited to a distinct area, e.g., outlined area, such as a patient’s chest or face. In some embodiments, the colormap of the background image is selected to be more neutral in color than the overlay of the physiological parameter. For example, the background image may have a colormap such as “bone,” “grey,” “pink,” “pastel,” while the overlay has more pronounced color(s), such as, e.g., red-green. Medical devices, e.g., tubing, leads, sensors, etc., may be shown in a different color or colormap.

Additionally, in some embodiments, the depth data used for the background image and/or the physiological parameter overlay may be less than the available data; the data used for the displayed image may be limited to a range more focused on the subject, e.g., the patient.

In some instances, even the enhanced image may appear “washed out” due to the colormap representing a broad range of depth or distance from the camera(s) (e.g., 400 mm to 1600 mm, or, 500 mm to 1800 mm, or, e.g., 200 mm to 2000 mm). However, a desired subject, such as the patient on the bed or the patient alone, is found in a much narrower distance range, e.g., 900 mm to 1300 mm, or, e.g., 900 to 1500 mm. Because of this, the desired subject is shaded or colored with a subset of the available colors of the colormap, with many colors not used or seen on the image. By applying the full colormap to a narrow depth range, the full range of colors can be seen in the image, resulting in the desired subject being more distinct from the surrounding background.

In order to use less than the full depth data, the desired data range must be selected. In some instances, the desired range may fluctuate, e.g., if a patient readjusts in bed; thus, the selected (narrow) range is preferably dynamically adjusted.

One example for selecting the range, for example when respiration is being monitored, is by determining regions in the field of view of the monitoring system that have active respiration and from those regions developing a mask that is applied to focus the respiratory monitoring and inhibit collection of data noise. The depth range within this mask would be used as the range, optionally with a margin applied to each end to better ensure all relevant surrounding areas are included. The margin could be predetermined (e.g., 200 mm on each end) or could be dynamic (e.g., 10% of the range added on each end). In another example, the range can be limited to all the depth data within the target regions, optionally with a margin. Alternately, the range may be set by the user. In another example, the largest and smallest values of the depth data could be used to set the range, which may be less than the monitored range (e.g., 400 mm to 1600 mm, etc.). After obtaining the narrowed range by any of these methods, the data may be filtered, so that the data changes slowly over time and does not cause artefacts in the displayed image.

Thus, described herein are methods and systems for improving or enhancing a visual output image from non-contact monitoring of a patient, by applying an adaptive histogram equalization transform, such as a contrast limited adaptive histogram equalization (CLAHE) transform, to depth data obtained from a non-contact monitoring system.

The above specification and examples provide a complete description of the structure and use of exemplary embodiments of the invention. The above description provides specific embodiments. It is to be understood that other embodiments are contemplated and may be made without departing from the scope or spirit of the present disclosure. The above detailed description, therefore, is not to be taken in a limiting sense. For example, elements or features of one example, embodiment or implementation may be applied to any other example, embodiment or implementation described herein to the extent such contents do not conflict. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

1. A method comprising:
receiving a video signal having depth data from a non-contact patient monitoring system;
enhancing a contrast of the depth data; and
applying a colormap to the enhanced contrast depth data to obtain an enhanced image.

2. The method of claim 1 wherein enhancing the contrast of the depth data comprises applying a histogram equalization transform to the depth data.

3. The method of claim 2, wherein the histogram equalization transform is a contrast limited adaptive histogram equalization (CLAHE) transform.

4. The method of claim 1 comprising:
selecting a depth data range,
enhancing a contrast of the depth data range; and
applying a colormap to the enhanced contrast depth data range.

5. The method of claim 1 further comprising:
determining an overlay of a monitored physiological parameter by the non-contact patient monitoring system; and
merging the enhanced image with the overlay.

6. The method of claim 5, wherein the monitored physiological parameter determined by the non-contact patient monitoring system is respiration.

7. A method comprising:
determining depth data between a non-contact patient monitoring system and a subject;
applying a histogram equalization transform to the depth data to obtain flattened depth data; and
applying a colormap to the flattened depth data to obtain an enhanced image.

8. The method of claim 7 further comprising:
selecting a depth data range from the depth data,
applying a histogram equalization transform to the depth data range to obtain flattened depth data range; and
applying a colormap to the flattened depth data range.

9. The method of claim 7 further comprising:
determining an overlay of a monitored physiological parameter by the non-contact patient monitoring system; and
merging the enhanced image with the overlay.

10. The method of claim 7, wherein the monitored physiological parameter determined by the non-contact patient monitoring system is respiration.

11. The method of claim 7, wherein the histogram equalization transform is a contrast limited adaptive histogram equalization (CLAHE) transform.

12. The method of claim 7, wherein the subject is a patient in a bed.

13. The method of claim 7, wherein the subject is an empty bed.

14. The method of claim 7, wherein the colormap is one of “bone,” “viridis,” “parula,” and “jet.”
